(19) United States
(12) Patent Application Publication
(10) Pub. No.: US 2016/0331961 A1
(43) Pub. Date: Nov. 17, 2016

(54) DEVICE FOR ELECTROSTIMULATION AND/OR IONTOPHORESIS

(71) Applicant: FEELIGREEN, Grasse (FR)

(72) Inventor: Christophe BIANCHI, Nice (FR)

(21) Appl. No.: 15/111,343

(22) PCT Filed: Jan. 14, 2015
(86) PCT No.: PCT/EP2015/050614

§ 371 Date: Jul. 13, 2016

(30) Foreign Application Priority Data
Jan. 14, 2014 (FR) 14 50258

(51) Int. Cl. A61N 1/32; A61M 37/00; A61N 1/04

(52) U.S. Cl. CPC A61N 1/325; A61N 1/0428; A61N 1/326; A61N 1/328; A61M 37/00; A61M 2037/0007

(57) ABSTRACT

A device for electrostimulation and/or iontophoresis. The device comprises a support having a first face intended to be placed opposite the skin and carrying electrodes configured so as to be in contact with the skin, and a second face opposite to the first, on which an electronic module is intended to be disposed, wherein the support comprises, on its first face, an electrical circuit connecting the electrodes, and a connector intended to connect the electrical circuit to the electronic module, and in that the device comprises a flexible partial-detachment mechanism carrying at least part of the connector and configured so as to make it possible to fold by flexing the connector beyond the first face towards the second face.

### TECHNICAL FIELD OF THE INVENTION

The present invention relates to a device for electrostimulation and/or iontophoresis.

The invention relates to techniques consisting of subjecting dermal cells to an electrical field in order to increase their activity and/or using an electric current for facilitating the transdermal diffusion of active substances, and relates more particularly to an electrostimulation and/or iontophoresis device.

Cell stimulation by an electrical field or electrostimulation is used in many applications. It is known for reducing pain or for cosmetic applications such as the treatment of wrinkles. For example, fibroblasts are subjected to an electrical field in order to increase their protein production rate. The proteins produced by fibroblasts being essentially collagen and elastin, the expected effect is an improvement in the elasticity of the skin and therefore a visible effect of wrinkle reduction.

Electrostimulation can also be used for treating the healing of wounds, where the electrical field increases cell migration and thus facilitates healing.

As for iontophoresis, this is a technique that can be used for cosmetic and/or medical purposes in order to introduce active substances into the dermis of the skin by means of a current.

### PRIOR ART

Unlike the traditional administration modes by oral or parenteral method, the duration of application of the current for iontophoresis or electrostimulation is lengthy. The current source must therefore be suitable for supplying a current for a long period. Furthermore, these devices also require means for controlling the current provided, for example, in the form of an electronic device able to be programmed according to the treatment or effect sought. The electronic device and the current source are associated with the element intended to be in contact with the skin. These devices are therefore often imposing through their size with a bulky current source and a bulky electronic device.

These devices are expensive and are thus reused for several patients, the elements being in contact with the skin then having to be cleaned or even sterilised in order to comply with health and safety rules. Moreover, in the context of iontophoretic use, it is often difficult to modify the active agent to be administered. This then requires a complete device for each active agent to be administered.

There therefore exists a need to propose a device that allows individual use or even single use while limiting costs.

### SUMMARY OF THE INVENTION

To this end the present invention concerns an electrostimulation or iontophoresis device comprising a support comprising a first face intended to be facing the skin and a second face opposite to the first. The first face comprises electrodes, an electrical circuit connecting the electrodes and connection means connecting the electric circuit to an electronic module. The device comprises flexible partial-detachment means carrying at least part of the connection means so that the connection means can be offset away from the first face of the support, preferentially at or beyond the second face of the support.

The device according to the invention affords ease of manufacture by forming all the conductive electrical elements on a single face of the support. The invention also allows simplified assembly since the electrical connection of the electrodes to the electronic module is obtained by flexing the partial-detachment means until they reach the electronic module disposed on the second face of the support.

Advantageously, the partial-detachment means form at least one tongue carrying the connection means. Preferentially, the electronic module is arranged between the at least one tongue offset at or beyond the second face or the support and said second face of the support. This arrangement has the advantage of easy and secure assembly of the electronic module on the support.

According to another aspect, the invention relates to a method for manufacturing an electrostimulation and/or iontophoresis device as described above comprising the following steps: producing, by depositing at least one layer of conductive material on a first face of an electrode support, an electrical circuit connecting the electrodes and connection means, advantageously depositing a layer of insulating material on the electrical circuit so as to electrically insulate it from the skin on which the device is intended to be applied, and forming means for partial detachment of the connection means. Preferentially, forming a cutout in the support in order to form means for partial detachment of the connection means. Preferentially, connecting an electronic module to the connection means at the second face of the support.

According to another aspect, the invention relates to a method for assembling the support and the electronic module of a device as described above by connecting partial-detachment means for bringing the connection means level with or beyond the second face of the support.

According to another independent aspect, the invention relates to a device for electrostimulation and/or iontophoresis, comprising a support having a first face intended to be placed facing the skin and a second face opposite to the first, an electronic module disposed on the second face of the support, characterised by the fact that: the support comprises electrodes on the first face, electrical and physical connection means intended to connect and fix the electronic module on the second face of the support, and an electrical circuit connecting the electrodes and the connection means, the support is a flexible surface larger than that of the electronic module, configured so as to conform to the areas of application to the human body to which it is intended to be applied.

The manufacturing method is therefore particularly simple and advantageously biocompatible.

### BRIEF DESCRIPTION OF THE FIGURES

The drawings are given by way of examples and are not limitative of the invention. They constitute outline schematic representations intended to facilitate understanding of the invention and are not necessarily to the scale of practical applications.

FIG. 1 depicts the support of the device according to the invention seen from the first face.

FIG. 2 depicts the support of the device according to the invention seen from the second face with the electronic module connected. The electrical circuit, the electrodes and the connection means are presented in solid lines for more clarity. However, they are arranged on the first face as described hereinafter.

FIG. 3 depicts a view in cross section of the device with the electronic module assembled on the support.

### DETAILED DESCRIPTION OF THE INVENTION

Before beginning a detailed review of the embodiments of the invention, optional features, which may optionally be used in association or alternatively, are stated below.

It is stated first of all that the invention relates to a device for electrostimulation and/or iontophoresis, comprising a support having a first face intended to be placed facing the skin and carrying electrodes configured so as to be in contact with the skin, and a second face opposite to the first, on which there is intended to be disposed an electronic module advantageously configured so as to supply a current to the electrodes and/or to control a current supply to the electrodes, characterised by the fact that the support comprises, on its first face, an electrical circuit connecting the electrodes and connection means intended to connect the electrical circuit to the electronic module, and in that the device comprises flexible partial-detachment means carrying at least part of the connection means and configured so as to make it possible to fold, by flexing, said connection means beyond the first face towards the second face, preferentially until they reach the electronic module.

Advantageously, the partial-detachment means form at least one tongue carrying the connection means so that flexing of the at least one tongue brings said connection means level with the first face or beyond in order to connect the electronic module.

Advantageously, the device comprising the electronic module is configured so as to be separable from the support.

Advantageously, the device comprising the electronic module is configured so as to be removable from the support.

Advantageously, the electronic module is in abutment on the second face of the support and is disposed between said second face of the support and the at least one tongue.

Advantageously, the partial-detachment means form at least two tongues.

Advantageously, two tongues folded by flexing beyond the first face level with or beyond the second face in order to connect the connection means to the electronic module exert a bearing force on said electronic module configured so as to press said electronic module on the second face of the support.

Advantageously, the electronic module is entirely supported by the second face of the support.

Advantageously, the connection means and the electronic module are configured so as to cooperate so that their cooperation provides simultaneously an electrical connection of the electronic module to the electrical circuit and physical holding of the electronic module on the second face of the support.

Advantageously, the connection means and the electronic module are configured so as to cooperate so that their cooperation provides by itself simultaneously an electrical connection of the electronic module to the electrical circuit and physical holding of the electronic module on the second face of the support.

Advantageously, the physical holding of the electronic module is solely provided by the connection means and the support.

Advantageously, the support is a flexible sheet with a surface area greater than that of the electronic module, configured so as to conform to the areas of application to the human body to which it is intended to be applied.

Advantageously, the surface area of the support is at least five times greater than that of the electronic module.

Advantageously, the support can be divided so as to be adaptable to the surface to which it is intended to be applied.

Advantageously, the device comprises a film of electrically insulating material intended to electrically insulate said electrical circuit from the skin to which the device is intended to be applied.

Advantageously, the electrical circuit, the electrodes and the connection means are formed by a conductive ink printed or screen printed on the first face of the support.

Advantageously, the support comprises at least one cosmetic or therapeutic active element disposed on its first face.

Advantageously, the support comprises a weft made from soaked biocellulose on the first face of the support of at least one active element.

Advantageously, the device comprises a textile weft covered on the first face of the support with a hydrogel containing at least one active element.

Advantageously, the support comprises an adhesive on its first face so as to keep the device applied on the skin.

Advantageously, the electronic module comprises a self-contained current source.

Advantageously, the support is for single use.

According to another aspect, the invention relates to a method for manufacturing an electrostimulation and/or iontophoresis device as described above, comprising the following steps: producing, by depositing at least one layer of conductive material on a first face of an electrode support, a printed circuit connecting the electrodes and connection means, deposition of a layer of an insulating material on the printed circuit so as to insulate it electrically from the skin on which the device is intended to be applied, formation by cutting of the means for partial detachment of the connection means.

According to another aspect, the invention relates to a method for assembling a device as described previously, comprising the flexing of the partial detachment means so as to bring the connection means level with or beyond the second face of the support and the connection of said connection means with the electronic module.

The invention relates to a device configured for electrostimulation and/or iontophoresis. The device comprises a support 1 comprising a first face 2a and a second face 2b opposite. Preferentially, the device comprises an electronic module 3.

The support 1 is intended to be applied to the skin by its first face 2a. The support 1 comprises electrodes 4 on the first face 2a so as to be in contact with the skin. These electrodes 4 make it possible to transmit an electric current to the skin. The electrodes 4 are advantageously flush with the surface of the first face. According to another possibility, the electrodes are in relief or recessed on the first face 2a.

“In contact” means a direct or indirect contact. In the case of iontophoresis, the first face 2a of the support 1 is not directly applied to the skin; the contact is indirect, for example by means of a layer containing active elements. Examples of layers and active elements are given earlier in the description. If the device is used as an electrostimulation device, the first face 2a of the support 1 is applied directly to the skin of the user without an intermediate layer containing active elements.

The device according to the invention is held in contact with the skin by fixing means. The fixing means are for example strips or threads that may be elastic and gripping the application zone. Preferably, the fixing means comprise an adhesive material applied to the first face 2a of the support 1 so as to hold the support 1 on the skin by means of the adhesive. Preferentially, the adhesive is an adhesive material on these two faces in order to cooperate with the first face 2a of the support and with the region of the skin on which the support is to be applied. The adhesive advantageously biocompatible, for example a dual-face hydrocolloid adhesive.

The support 1 comprises a printed electrical circuit 6 intended to connect the electrodes 4. Advantageously, the printed circuit 6 is disposed on the first face 2a of the support 1. Preferentially, the printed circuit 6 is electrically insulated from the skin on which the device is intended to be applied. By way of example, a layer of insulating material is applied to the printed circuit 6. The layer of insulating material excludes the electrodes 4. Preferentially, the layer of insulating material excludes the connection means 5.

The support 1 comprises connection means 5 intended to connect the electronic module 3 to the printed circuit 6.

According to an advantageous embodiment, the printed circuit 6, the electrodes 4 and the connection means 5 are formed on the first face 2a of the support 1. This allows easy manufacture with deposits of electronic layers on a single face of the support 1.

By way of example, the electrodes 4, the printed circuit 6 and the connection means 5 are formed by printing with a conductive ink on the support 1. By way of example, conductive inks based on silver, gold or zinc oxide may be used. Preferentially, the electrodes 4 intended to be in contact with the user are made from a bioconductive material such as for example PEDOT-PSS (poly(3,4-ethylenedioxidethiophene)-poly(styrenesulfonate). The printed electrical circuit 6 may also be made from conductive fibres woven in a textile.

According to the invention, the support 1 advantageously comprises partial-detachment means 7 carrying at least part of the connection means 5. The partial-detachment means 7 are intended to enable the connection means 5 formed on the first face 2a of the support 1 to be moved level with the second face 2b and preferentially beyond the second face 2.

Preferentially, the first face 2a defines a plane in which the electrodes 4 and the printed circuit 6 are arranged. The connection means 5 are configured so as to be able to be arranged in the plane of the first face 2a and outside it, preferentially beyond the second face 2b, when the connection means 5 are active. Active means that the connection means 5 are connected to the electronic module 3. Conversely, the connection means 5 are said to be inactive when they are not connected to the electronic module 3.

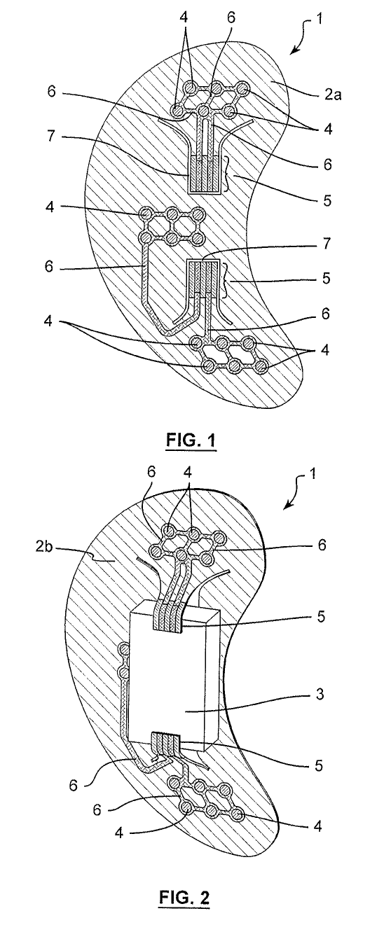

According to one embodiment, the partial-detachment means 7 are formed by cutouts on the support 4 around the connection means 5. The partial-detachment means 7 define at least one tongue, preferentially two tongues. Each tongue at least partially carries the connection means 5. The partial-detachment means 7 are flexible so as to be flexed and offset outside the first face 2a, preferentially level with the second face 2b or beyond. The cutouts are arranged for example as depicted in FIGS. 1 to 3. The partial-detachment means 7, preferentially each tongue comprises a connection zone providing the electrical connection between the printed circuit 6 and the connection means 5. The connection with the printed circuit 6 is maintained. The free end of the partial-detachment means 7, preferentially opposite to the connection zone and therefore the zone for connection to the printed circuit 6, advantageously comprises the connection means 5 intended to be connected to the electronic module 3. This free end is able to move with respect to the support 1.

This arrangement allows manufacture on a single face of the support 1 while allowing connection to the electronic module 3 on the opposite face.

According to a preferred embodiment of the invention, the connection means 5 are disposed in the plane of the first face 2a of the support 1 when they are not connected to the electronic module 3 and are disposed outside the plane of the first face 2a of the support 1 when they are connected to the electronic module 3.

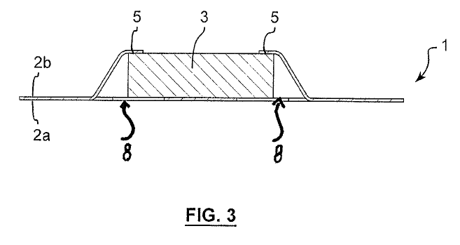

Preferably, the connection means 5 are simultaneously electrical and physical connection means. That is to say the connection means 5 provide an electrical connection of the electronic module 3 and of the printed circuit 6 and simultaneously they provide physical fixing of the electronic module 3 on the support 1. Preferentially, as shown in FIG. 3, the electronic module 3 is in abutment on the second face 2b of the support and the connection means 5 come to be applied on complementary zones of the electronic module 3. The electronic module 3 is disposed between the second face 2b of the support 1 and at least one tongue, preferentially the two tongues. Preferentially, the connection means 5, or more precisely the tongues defined by the partial-detachment means 7, are facing each other so as to hold the electronic module 3. The electronic module 3 is supported by the support 1, that is to say the support 1 opposes the fall by gravity of the electronic module 3. Advantageously, the electronic module 3 is in abutment through its entire surface arranged opposite the second face 2b.

The detachment of the tongues beyond the first face 2a towards or beyond the second face 2b opposes a return force. In this way, when the tongues are arranged above the electronic module 3 and the connection means 5 cooperate with the electronic module 3, they exert an abutment force on the electronic module 3 so as to press it on the second face 2b of the support 1. By way of example, the tongues have a length of 1 to 3 cm.

According to an advantageous embodiment, the electronic module 3 comprises means for measuring physiological parameters of the user. Preferentially, at least one sensor, for example for measuring temperature, is arranged on the face of the electronic module 3 opposite the second face 2b of the support 1 at the opening 8 formed by the movement of the connection means at the second face 2b of the support 1 at the opening 8 formed by the movement of the connection means at the second face 2b of the support 1 so as to be connected to the electronic module 3. In this way, the sensor is arranged substantially level with the first face 2a of the support 1 and can therefore be in contact with the skin once the device is applied to the user.

The electronic module 3 is advantageously intended to control the current supplied. The electronic module 3 advantageously makes it possible to control the intensity, the voltage and/or the duration of administration. According to one possibility, the electronic module 3 comprises a microprocessor supplied by an electrical energy source such as a rechargeable accumulator or a battery and which controls the application of a voltage to the electrodes 4, for example by means of a voltage generator.

The energy source may be of various types. Advantageously, the energy source is self-contained, by way of example a self-contained energy generator working by recovery of energy may be used, or batteries of the cell type.

According to an advantageous embodiment, the electronic module 3 comprises the current source, preferentially a self-contained current source. This arrangement is particularly advantageous for allowing ambulatory use of the device according to the invention.

According to a preferred embodiment, the electronic module 3 can be separated from the support 1. More precisely, the electronic module 3 can be removed from the support 1. Preferentially, the electronic module 3 can be reused. The support 1 for its part is advantageously a consumable that will be used only once or a few times but to a lesser extent than the electronic module 3. The support 1 is disposable. This is particularly advantageous from an ecological and economic point of view since the support 1 comprises the electrodes 4. However, the applicant has found that the electrodes of the electrostimulation and/or iontophoresis devices suffered strong oxidation and therefore damage over time. This arrangement makes it possible to dispose of the support 1 after use without disposing of the electronic part, which is more expensive. In addition, it is possible, with a single electronic module 3, to administer a plurality of active substances of to carry out alternately electrostimulation and iontophoresis solely by changing the support 1.

The device according to the invention makes it possible to have a device that may be for single use or at least used for a single patient while reusing the expensive electronic elements.

According to the invention, the support 1, which may also be referred to as a patch, is a flexible cloth. The flexibility is such that two opposite points on the periphery may be pressed against each other without damage to the support 1. This flexibility is advantageously maintained when the electronic module 3 is connected and fixed to the support 1. Preferentially, the support 1 has a thickness of 40 to 60 μm. the flexibility of the support 1 enables the device to be conformed to the area of application to the body where it is intended to be applied.

The device according to the invention can be used on all areas of the human body easily and without any trouble or inconvenience for the user.

The support 1 has a surface area greater than that of the electronic module 3. More precisely, the surface area of the first face 2a of the support 1 is larger than the surface area of the electronic module 3 facing the support 1. Advantageously, the ratio of the surface areas of the first face 2a and electronic module 3 is at least equal to ⅕. This difference in size makes it possible in particular to maintain flexibility of the support for a good application of the entire area to be treated.

Preferentially, the electronic module 3 has a surface area opposite the support 1 of around 1 to 6 cm², preferentially the first face 2a of the support 1 is around 8 to 50 cm².

By way of example, the support 1 has a thickness of 50 μm and dimensions of 41 mm wide and 142 mm long.

The surface of the support 1 can advantageously be adapted to the surface on which it is intended to be applied. By way of example, the support 1 comprises a central portion, and at least one broadened portion carrying at least two electrodes extends on either side of the central portion. Advantageously, the support 1 can be divided in order to adapt to the surface on which it is intended to be applied. In an embodiment in which the support 1 comprises two broadened portions extending on either side of the central portion, the support 1 may be divided between two broadened portions in order to reduce the application surface of the support 1. The flexibility of the support 1 advantageously allows this cutting. Dividable means that the support 1 is configured so as to be cut or divided even at the electrical circuit 6.

The device according to the invention can be used in cosmetic or therapeutic applications. For example, if the device is used for electrostimulation, the current penetrates the skin in order either to stimulate the fibroblasts so as to increase the collagen and elastin production rate thereof, the effect of which to improve the elasticity of the skin, or to treat healing of a wound. If the device is used for iontophoresis, it is intended to make active substances enter the dermis, such as for example vitamin A or retinol having a depigmentation effect, retinoic acid for treating acne, vitamin C having an antioxidant effect, an ion chelator such as β-alanine di-acetic acid for treating erythema, glycolic acid improving skin texture, dexamethasone sodium phosphate having an anti-inflammatory effect or any other type of active agent either ionised or presented in the form of anionic or cationic emulsions in order to be able to be conveyed by the current.

The electronic module 3 is configured to deliver a current of low voltage and limited density. Preferably, the voltage U is less than or equal to 1 V and the current I is less than or equal to 1 mA.

Preferentially, the support 1 comprises a plastics material. By way of example, the support 1 is made from polyimide (PI), polyethylene terephthalate (PET), polyether ether ketone (PEEK) or polyethylene naphthalate (PEN).

According to another embodiment, the support 1 comprises a textile weft, for example nylon.

The material of the support 1 is preferably thin. The material of the support 1 is preferentially resistant to high temperatures for any curing of the conductive ink.

The support 1 is applied alone for electrostimulation or with an intermediate layer for iontophoresis.

According to one embodiment, the device comprises biocellulose, preferably soaked for example with active substances for iontophoresis.

According to another embodiment, the device comprises a gel containing active substances for iontophoresis. A gel may be applied directly to the skin and then the support 1 is applied on top or the gel is applied to the skin and the support 1 is applied on top or the gel is applied to the support 1 and then the whole is next applied to the skin. The gel may form the means for fixing the support 1 to the skin, in particular by having adhesion properties.

By way of non-limitative example, the adhesive-type fixing means and/or the intermediate layer containing active substances measure from 25 to 60 μm thick.

The electronic module 3 comprises, according to possibilities, sensors for physiological parameters such as temperature, pH or resistivity, and/or indicator lights such as LEDs (light emitting diodes).

According to an advantageous possibility, the device comprises a plurality of anodes on a plurality of cathodes alternating with the anodes. In this configuration, the total current is divided into a plurality of anode-cathode circuits so that the density of the current flowing from an anode to a cathode is preferentially less than 1 mA/cm². Furthermore, in order to prevent the formation of external field lines, that is to say outside the device, the two electrodes situated at the two ends have the same polarity, that is to say it is two anodes or two cathodes.

### REFERENCES

1 Support
2a. First face
2b. Second face
3 Electronic module
4 Electrodes
5 Connection means
6 Printed electrical circuit
7 Partial-detachment means
8 Opening

1. A device for electrostimulation and/or iontophoresis, comprising a support having a first face intended to be placed opposite the skin and carrying electrodes configured so as to be in contact with the skin, and a second face opposite to the first, on which an electronic module is intended to be disposed, wherein
the electronic module is configured so as to be removable from the support, wherein the support comprises, on its first face, an electrical circuit connecting the electrodes and connection means intended to connect the electrical circuit to the electronic module, and wherein the support comprises flexible partial-detachment means forming at least one tongue carrying the connection means and configured so as to make it possible to fold by flexing the at least one tongue in order to bring said connection means beyond the first face level with or beyond the second face in order to connect the electronic module.

2. The device according to claim 1, wherein the electronic module is configured so as to be separable from the support.

3. The device according to claim 1, wherein the electronic module is in abutment on the second face of the support and is disposed between said second face of the support and the at least one tongue.

4. The device according to claim 1, wherein the partial-detachment means form at least two tongues.

5. The device according to claim 4, wherein the two tongues folded by flexing beyond the first face level with or beyond the second face in order to connect the connection means to the electronic module exert an abutment force on said electronic module configured so as to press said electronic module on the second face of the support.

6. The device according to claim 5, wherein the electronic module is entirely supported by the second face of the support.

7. The device according to claim 1, wherein the connection means and the electronic module are configured so as to cooperate so that their cooperation simultaneously provides an electrical connection of the electronic module to the electrical circuit and physical holding of the electronic module on the second face of the support.

8. The device according to claim 1, wherein the connection means and the electronic module are configured so as to cooperate so that their cooperation provides by itself simultaneously an electrical connection of the electric module to the electrical circuit and physical holding of the electronic module on the second face of the support.

9. The device according to claim 8, wherein the physical holding of the electronic module is solely provided by the connection means and the support.

10. The device according to claim 1, wherein the support is a flexible sheet with a surface area greater than that of the electronic module, configured so as to conform to the areas of application on the human body to which it is intended to be applied.

11. The device according to claim 10, wherein the surface area of the support is at least five times greater than that of the electronic module.

12. The device according to claim 1, wherein the support is dividable so as to be adaptable to the surface on which it is intended to be applied.

13. The device according to claim 1, comprising a film of electrically insulating material intended to electrically insulate said electrical circuit from the skin on which the device is intended to be applied.

14. The device according to claim 1, wherein the electrical circuit, the electrodes and the connection mean are formed by a conductive ink printed or screen printed on the first face of the support.

15. The device according to claim 1, wherein the support comprises at least one cosmetic or therapeutic active element disposed on its first face.

16. The device according to claim 15, wherein the support comprises a biocellulose weft soaked on the first face of the support with at least one active element.

17. The device according to claim 15, wherein the support comprises a textile weft covered on the first face of the support with a hydrogel containing at least one active element.

18. The device according to claim 1, wherein the support comprises an adhesive on its first face so as to keep the device applied on the skin.

19. The device according to claim 1, wherein the electronic module comprises a self-contained current source.

20. The device according to claim 1, wherein the support is for single use.

21. The method for manufacturing an electrostimulation and/or iontophoresis device according to claim 1, comprising the following steps: producing, by depositing at least one layer of conductive material on a first face of a support of electrodes, an electrical circuit connecting the electrodes and connection means, depositing a layer of insulating material on the electrical circuit so as to electrically insulate it from the skin on which the device is intended to be applied, and forming, by cutting, means for partial detachment of the connection means.

22. The method for assembling a device according to claim 3, comprising the flexing of the partial-detachment means so as to bring the connection means level with or beyond the second face of the support and the connection of said connection means to the electronic module.
